(19) United States
(12) Patent Application Publication
(10) Pub. No.: US 2021/0145297 A1
(43) Pub. Date: May 20, 2021

(54) BLOOD FLOW MEASURING APPARATUS AND METHOD HAVING FUNCTION OF CORRECTING NOISE DUE TO PRESSURE

(71) Applicants: CMLAB CO., LTD., Seoul (KR); KOREA UNIVERSITY RESEARCH AND BUSINESS FOUNDATION, Seoul (KR)

(72) Inventors: Beop-Min KIM, Seoul (KR); Tae Ho HA, Goyang (KR); Seung Ho PAIK, Seongnam (KR)

(73) Assignees: CmLab Co., Ltd., Seoul (KR); Korea University Research and Business Foundation, Seoul (KR)

(21) Appl. No.: 17/043,533

(22) PCT Filed: Mar. 30, 2018
(86) PCT No.: PCT/KR2018/003802

§ 371 Date: Sep. 29, 2020

(51) Int. Cl. A61B 5/026; A61B 5/00; A61B 5/1455

(52) U.S. Cl. CPC A61B 5/0261; A61B 5/7203; A61B 5/1455; A61B 2562/0247; A61B 5/4866; A61B 5/7278; A61B 5/6843

(57) ABSTRACT

The present invention relates to a blood flow measuring apparatus and method having a function of correcting noise due to pressure, the apparatus comprising: a light source module configured to come into contact with tissue so as to emit light thereat; a detection module for detecting the light emitted at the tissue; a pressure measurement module configured to measure pressure applied to the tissue; and a signal processing unit for processing a signal received from the detection module by applying a correction function to the signal such that noise, which is generated according to the pressure applied when light is detected, can be corrected. A blood flow measuring apparatus and method having a function of correcting noise due to pressure can improve accuracy and reliability since the effect of noise, which is generated according to a change in pressure, can be removed from detected data.

### TECHNICAL FIELD

Research related to this patent was made with the support of Brain Science Fundamental Technology Development Project (Project name: Research for analysis of structural and functional disorders in brain development and development of diagnostic equipment, and Project serial No. 1711057527) under the supervision of Korea Ministry of Science and ICT.

The present disclosure relates to a blood flow measuring apparatus and method having a function of correcting noise due to pressure and, more particularly, to a blood flow measuring apparatus and method having a function of correcting noise due to pressure when a change in blood flow is detected in a noninvasive manner using light.

### BACKGROUND ART

Recently, as a method of acquiring bio-information, research into near-infrared spectroscopy (NIRS) is being actively conducted. The NIRS is a method in which a body tissue may be imaged using light harmless to the human body. This can minimize cost burdens unlike other methods. As the related art, Korean Patent No. 1,008,041 has been disclosed.

However, the conventional method of measuring a change in blood flow using the NIRS is problematic in that it is difficult to obtain accurate data when a subject is out of a stable state, for example, pressure is increased by external force.

### DISCLOSURE

### Technical Problem

An objective of the present disclosure is to provide a blood flow measuring apparatus and method, capable of improving accuracy by removing noise caused by the application of pressure from data acquired when pressure in tissue is changed.

### Technical Solution

The present disclosure may provide a blood flow measuring apparatus having a function of correcting noise due to pressure, including a light source module configured to come into contact with tissue to emit light; a detection module configured to detect light that has undergone at least one process of scattering, absorption, and reflection while moving along the tissue; a pressure measurement module configured to measure pressure acting on the tissue; and a signal processing unit configured to process a signal received from the detection module by applying a correction function to correct noise generated as the pressure acts during the detection of the light.

The signal processing unit may correct the signal received from the detection module using the correction function due to the pressure.

The signal processing unit may perform correction using a correction value matching with the pressure from a correction value table.

The correction function may be acquired by interpolating detection optical data acquired when the pressure is changed in a state where light is emitted to the tissue with the light source module.

The correction function may be acquired by interpolating detection optical data acquired by changing pressure when two types of light having different wavelengths are emitted to the tissue.

The light may be selected in a near-infrared area, and the signal processing unit transforms data on a light intensity into Oxy hemoglobin concentration and Deoxy hemoglobin concentration to grasp metabolism of the tissue on the basis of the detection optical data.

The signal processing unit may perform interpolation through transformation of at least one of variance for an initial measurement value or log scale so that change tendencies depending on a change in pressure are synchronized for values detecting light having different wavelengths.

The correction function may deduce −log(Pressure/Po) value from the pressure data, deduce −log(Intensity/Io) value from the detection optical data, and be determined by interpolation according to distribution of the −log(Pressure/Po) value and the −log(Intensity/Io) value.

The correction function may acquire the −log(Pressure/Po) value and the −log(Intensity/Io) value by linear interpolation or quadratic interpolation.

The correction function may be calculated on the basis of detection data acquired according to pressure in a state where the light is emitted to a phantom.

The light source module, the detection module, and the pressure measurement module may constitute one probe, and a plurality of probes may be connected to each other to surround a head.

The light source module, the detection module, and the pressure measurement module may be provided on a surface of the probe to come into contact with the tissue.

The signal processing unit may determine a value acquired from the detection module as an error value, when a value measured in the pressure measurement module is equal to or less than a reference value.

Furthermore, the present disclosure may provide a blood flow measuring method having a function of correcting noise due to pressure, including a step of emitting light while coming into contact with tissue; a step of acquiring detection optical data by detecting light that has undergone at least one process of scattering, absorption, and reflection while moving along the tissue; a step of measuring pressure acting on the tissue; and a correcting step of reflecting a correction function depending on the pressure to the detection optical data so as to remove noise of the detection optical data due to the pressure.

The correction function may be calculated by performing a correction-function generating step, and the correction-function generating step is acquired by performing: a test-light emitting step of emitting light to the tissue; a step of changing pressure applied to the tissue; a step of acquiring test detection optical data according to a change in the pressure; and an interpolation step of performing interpolation from the detection optical data and the pressure data.

The interpolation step may deduce −log(Pressure/Po) value from the pressure data, deduce −log(Intensity/Io) value from the detection optical data, and perform interpolation according to distribution of the −log(Pressure/Po) value and the −log(Intensity/Io) value.

The interpolation step may determine the −log(Pressure/Po) value and the −log(Intensity/Io) value with the correction function acquired by linear interpolation or quadratic interpolation.

The step of emitting light may use light in a near-infrared area, and the method may further include, after the correcting step, a signal processing step of transforming data on a light intensity into Oxy hemoglobin concentration and Deoxy hemoglobin concentration to grasp metabolism of the tissue on the basis of the detection optical data.

The correction function may be calculated by performing a correction-function generating step, and be calculated by performing: a test-light emitting step of emitting light to a phantom; a step of changing pressure applied to the phantom; a step of acquiring test detection optical data according to a change in the pressure; and an interpolation step of performing interpolation from the detection optical data and the pressure data.

The blood flow measuring method may further include an error-value classifying step of determining the detection optical data as an error value, when a measured pressure value is less than a reference value.

### Advantageous Effects

A blood flow measuring apparatus and method having a function of correcting noise due to pressure according to the present disclosure can remove the effects of noise caused by a change in pressure from detected data, thus improving accuracy and reliability.

### DESCRIPTION OF DRAWINGS

FIG. 1 is a block diagram illustrating the configuration of a blood flow measuring apparatus in accordance with a first embodiment of the present disclosure.

FIG. 2 is a diagram illustrating the use of the first embodiment.

FIG. 3 is a perspective view illustrating an installation part of the blood flow measuring apparatus.

FIG. 4a is a sectional view illustrating a probe of the blood flow measuring apparatus.

FIG. 4b is a bottom view illustrating the probe of the blood flow measuring apparatus.

FIG. 5 is a sectional view illustrating a concept in which the probe is applied to a phantom.

FIG. 6 is a diagram illustrating detection data as a function of a change in pressure.

FIG. 7 is a graph in which values of FIG. 6 are converted into LOG SCALE.

FIG. 8 is a diagram in which values of FIG. 7 are scaled.

FIG. 9 is a graph illustrating a result obtained by linear interpolation in FIG. 8.

FIG. 10 is a graph illustrating a result of performing correction when a light source having the wavelength of 760 nm is used.

FIG. 11 is a graph illustrating a result of performing correction when a light source having the wavelength of 830 nm is used.

FIG. 12 is a graph before and after the correction of Oxy and Deoxy values.

FIG. 13 is a graph illustrating detection values before and after correction when interpolation is performed according to a quadratic interpolating polynominal.

FIG. 14 is a flowchart illustrating a blood flow measuring method in accordance with another embodiment of the present disclosure.

FIG. 15 is a detailed flowchart illustrating a step of generating a correction function.

FIG. 16 is a flowchart illustrating a blood flow measuring method in accordance with another embodiment of the present disclosure.

### MODE FOR DISCLOSURE

Hereinafter, a blood flow measuring apparatus and method having a noise correcting function due to pressure in accordance with an embodiment of the present disclosure will be described in detail with reference to the accompanying drawings. Furthermore, in the description of the following embodiments, the terms of respective components will be referred to as different terms in the art. However, as long as the components have functional similarity or identity, it can be seen as having the same configuration even if a modified embodiment is adopted. Furthermore, reference numerals added to respective components are described for the convenience of description. However, respective components denoted by reference numerals and shown in the drawings are not limited to the scope of the drawings. Likewise, even if an embodiment having components of the drawings that are partially modified is adopted, it can be seen that the embodiment has the same configuration as long as the components have functional similarity or identity. Furthermore, if those skilled in the art can easily understand components, a description thereof will be omitted.

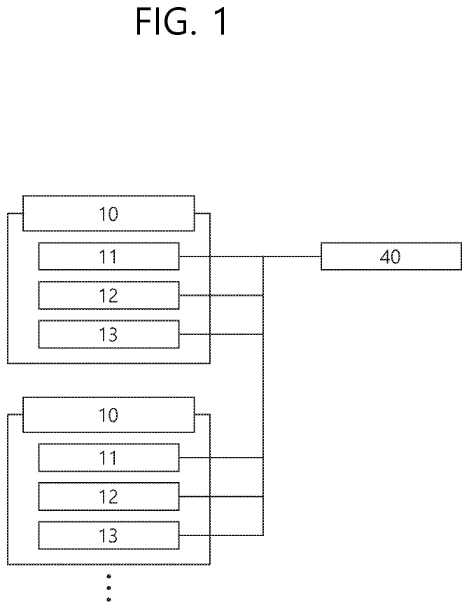

Hereinafter, the configuration of a blood flow measuring apparatus 100 in accordance with a first embodiment of the present disclosure will be described in detail with reference to FIGS. 1 to 4.

The blood flow measuring apparatus 100 may be provided with a plurality of probes to measure blood flow at a plurality of points, and may be provided with a light source module 11, a detection module 12, and a pressure measurement module 13 which are provided in each probe. A signal processing unit 40 may be configured to control the light source module 11 and to process a signal on the basis of a signal received from the pressure measurement module 13 and the detection module 12.

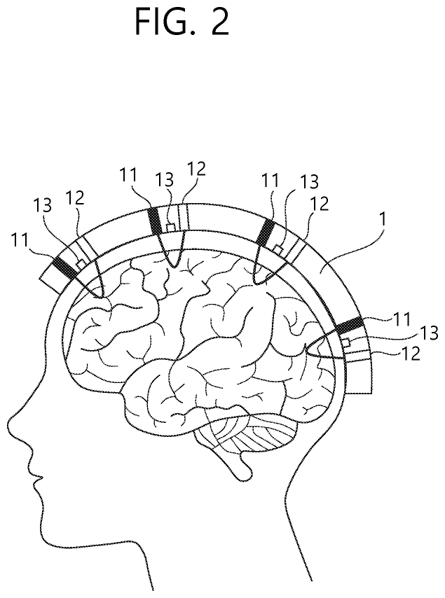

FIG. 2 is a diagram illustrating the use of the blood flow measuring apparatus in accordance with the first embodiment of the present disclosure. The blood flow measuring apparatus is worn on a body to come into close contact with skin tissue, and is configured to measure the blood flow in a noninvasive manner. For instance, the measuring apparatus is configured in the shape of a helmet that is worn on a person's head to measure cerebral blood flow using NIRS. The NIRS is a method in which a change in concentration of an absorbing substance such as oxidized hemoglobin, reduced hemoglobin, or myoglobin present in the body tissue and an optical coefficient thereof may be measured in a noninvasive manner. Since near-infrared rays in the band of 700 to 2800 nm, particularly 700 to 900 nm are relatively smaller in scattering and absorption in the body tissue compared to those of other bands, light may reach deeply. Based on this fact, it is possible to obtain information up to the depth of several centimeters in the body. The absorbing substance present in the body may be largely divided into a substance that is dependent on oxygen and a substance that is not dependent on oxygen. In particular, since a change in concentration of the substance that is dependent on oxygen is closely related to metabolic activity in the body, it is very important to quantitatively and qualitatively analyze the change in concentration. The metabolism of tissue may be understood by calculating oxy hemoglobin concentration (Oxy) and deoxy hemoglobin concentration (Deoxy) using an algorithm for deducing a significant analyzed result based on the detected optical signal.

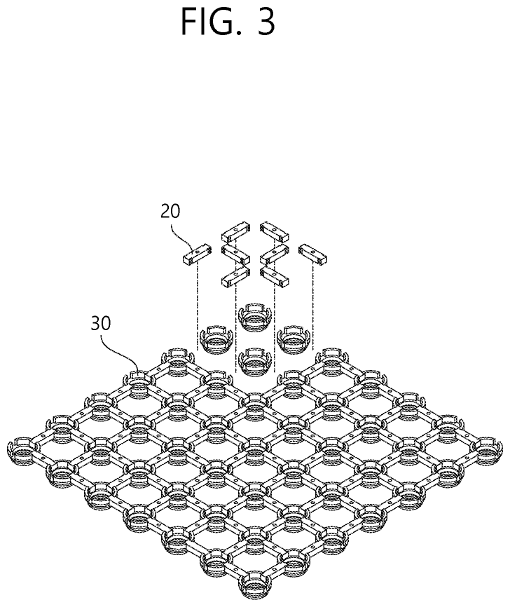

FIG. 3 is a perspective view illustrating an installation part 1 of the blood flow measuring apparatus 100. As shown in the drawing, the installation part 1 of the blood flow measuring apparatus may be configured by combining a connecting module 20 and a receiving module 30. The connecting module 20 is configured to extend a predetermined length in a longitudinal direction, and is provided with a fastening part formed of a groove or a protrusion to allow both ends of the connecting module to be coupled to adjacent receiving modules 30. The receiving module 30 may be formed of a protrusion or a groove to be mechanically fastened to a corresponding coupling part of the connecting module 20. A probe 10 capable of measuring blood flow that will be described below may be fastened to the receiving module 30.

The installation part 1 may be configured in the shape of a mesh to measure cerebral blood flow at a plurality of points, and may be provided with a module capable of measuring blood flow at each node. Meanwhile, the blood flow measuring apparatus is changed in shape to come into close contact with various body parts. A user may configure the blood flow measuring apparatus by selectively combining modules according to a body part on which the apparatus is to be worn.

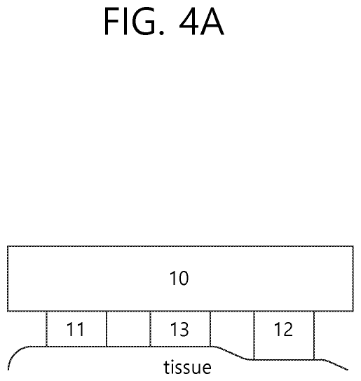

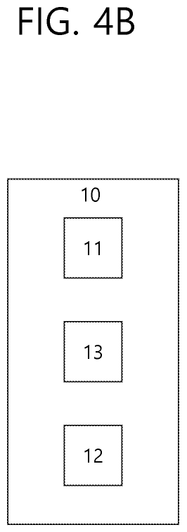

FIG. 4a is a sectional view illustrating the probe 10 of the blood flow measuring apparatus, and FIG. 4b is a bottom view illustrating the probe 10 of the blood flow measuring apparatus 100. As shown in the drawings, the blood flow measuring apparatus 100 using the NIRS is configured to include the light source module 11, the detection module 12, the pressure measurement module 13, and the probe 10 including the above-described components, and the signal processing unit 40. The light source module 11, the detection module 12, and the pressure measurement module 13 are provided on a surface of the probe 10 while being in contact with a site of a subject that is to be examined.

The probe 10 functions as a unit module for measuring blood flow. As described above, the probe 10 may be configured to include the light source module 11, the pressure measurement module 13, and the detection module 12, may be configured to come into close contact with the body, and may be configured to be connected to and supported by the above-described installation part 1.

The light source module 11 emits light to a site that is to be examined. The light emitted to the site is scattered, absorbed, and reflected while moving through the body, thus reaching the detection module 12. Therefore, light reaching the detection module 12 includes various pieces of bio-information such as information about blood flow in the body, and may measure the blood flow in the body on the basis of the information. The light source module 11 may be composed of an LED, and be configured to generate light having an inherent wavelength. As described above, the inherent wavelength may be light having a near-infrared wavelength. The light source module 11 may be configured to include a plurality of light sources that generate light having a wavelength.

The detection module 12 is configured to detect light in which near-infrared rays have undergone at least one of scattering, absorption, and reflection in tissue. The detection module 12 is provided to be spaced apart from the light source module 11 by a proper distance. In the case of obtaining information about a deep portion in the tissue, a distance between the light source module 11 and the detection module 12 may be increased. The detection module 12 is configured to detect near-infrared rays generated from the light source module 11. For instance, the detection module may be configured as a photodiode.

The pressure measurement module 13 is configured to measure pressure acting on tissue that is to be examined. A value measured by the pressure measurement module 13 is measured so as to correct noise due to pressure, and is also used to determine whether the probe 10 is properly attached to a part that is to be examined.

However, the above-described probe 10 may be changed in various shapes to come into contact with the part that is to be examined.

The signal processing unit 40 is configured to control the light source module 11 and to receive a signal from the detection module 12 and the pressure measurement module 13. The signal processing unit 40 may previously set a correction function and thus perform a function of removing noise from detection data acquired from the detection module 12. The correction function may be set in a predetermined range, and may be configured so that a user may select the range according to a patient's condition or environment. Furthermore, the signal processing unit 40 may be configured to include an image display algorithm for performing a display in a form that is easily recognized by a user according to a value measured by the detection module 12. Meanwhile, the function of correcting noise due to pressure by the signal processing unit 40 will be described below in detail.

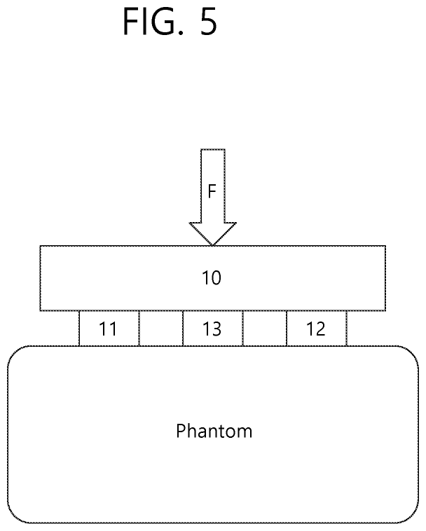

FIG. 5 is a sectional view illustrating a concept in which the probe 10 is applied to a phantom. In the present disclosure, in order to correct noise due to pressure, the quantity of light is measured in environment where pressure is artificially applied, and the correction function is calculated by interpolation. The phantom means a model used in place of the body for biotrepy such as investigation or analysis of the electromagnetic-wave distribution in the body and the specific absorption rate (SAR) of the body tissue. The phantom has a shape similar to that of the body tissue, and is configured to have similar specific dielectric constant, conductivity, and density. Here, a pressure sensor is configured to come into contact with the phantom between the light source module 11 and the detection module 12, and is configured to measure pressure generated by force acting between the phantom and the probe 10. However, although the pressure measurement module 13 is shown in a configuration where it directly comes into close contact with the phantom, the pressure measurement module may be provided at various positions as a position for measuring pressure by external force.

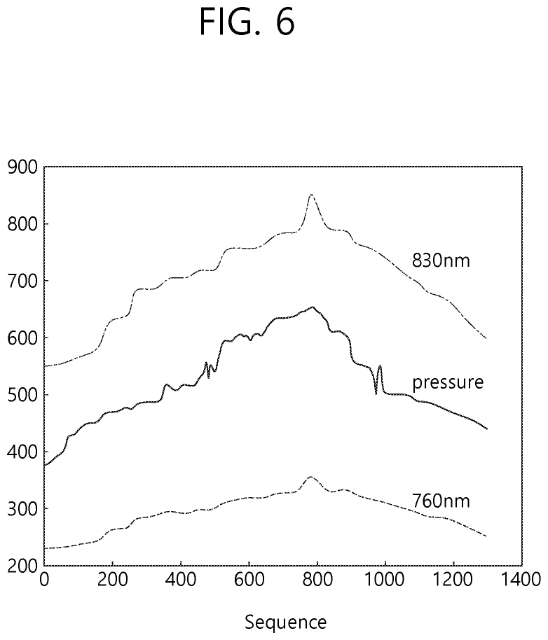

Hereinafter, the generation of the correction function used to correct noise due to pressure will be described in detail for the phantom with reference to FIGS. 6 to 9.

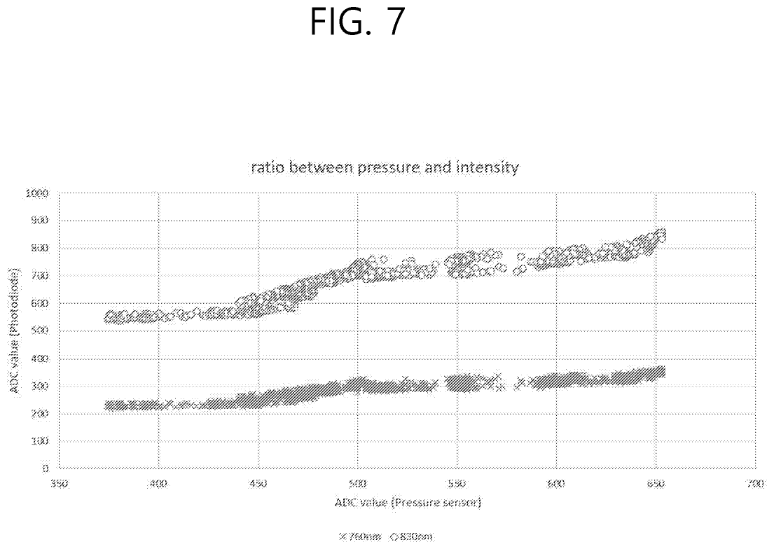

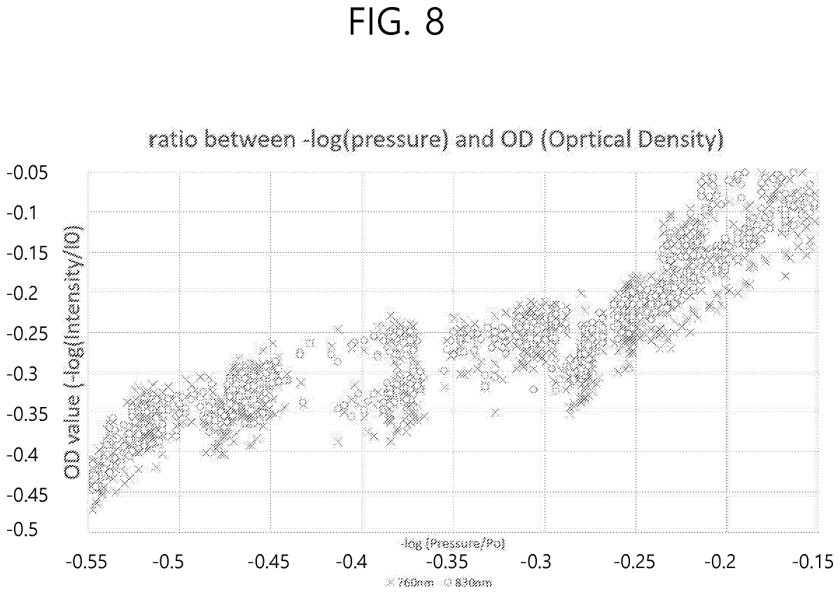

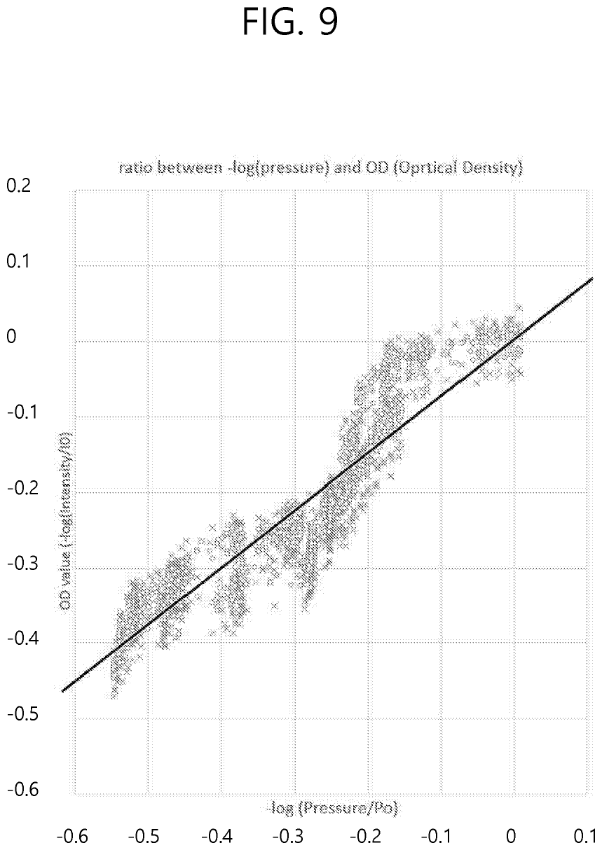

FIG. 6 is a diagram illustrating detection data as a function of a change in pressure, FIG. 7 is a graph in which values of FIG. 5 are converted into LOG SCALE, FIG. 8 is a diagram in which values of FIG. 7 are scaled, and FIG. 9 is a graph illustrating a result obtained by linear interpolation in FIG. 8.

In order to obtain the correction function using the phantom, the probe is first in close contact with the phantom, like when it is used in actual tissue. Subsequently, the light source module is operated to continuously emit light having a specific wavelength to the phantom. The pressure is changed by changing force acting on the probe while maintaining the operation of the light source module. The light source module 11 uses the light source module 11 having a first wavelength and the light source having a second wavelength to obtain the result according to the wavelength. The detection module 12 measures the intensity of light that varies with the change of pressure.

In FIGS. 6 to 9, the first wavelength is selected to be 760 nm, and the second wavelength is selected to be 830 nm. The probe 10 is configured independently for two types of light having different wavelengths, so that data is acquired and the processed results are simultaneously displayed.

FIG. 6 shows pressure applied to the phantom according to a sequence and a value measuring the intensity of acquired light as an ADC (Analog to Digital converter) value. The pressure application sequence is performed using the profile of pressure acting on the probe 10 according to a time sequence included in pressure data. FIG. 6 shows first detection optical data acquired from the detection module 12 when light of the first wavelength is emitted according to the same pressure application profile, and second detection optical data acquired from the detection module 12 when light of the second wavelength is emitted. It can be seen that the intensity value of the acquired light when the wavelength of light is 830 nm is higher than that when the wavelength is 760 nm. Since physical properties indicating metabolism-related information included in the blood flow are not changed even if pressure acting on the phantom is changed, it is preferable to acquire the intensity value of light regardless of pressure. However, a change in the ADC value depending on a change in pressure shows a similar tendency, which means that noise due to pressure is included and thus an incorrect value is generated.

FIG. 7 is a graph showing, as the ADC value, the intensity of light due to a change in pressure using the acquired detection optical data. As shown in the drawing, when light having the wavelength of 760 nm and light having the wavelength of 830 nm are emitted, it can be seen that the detected ADC value generally increases in a similar manner as pressure increases. However, the step of changing and applying a scale is required to check whether the detection optical data has the same tendency.

Referring to FIG. 8, when the wavelengths of light are 760 nm and 830 nm, respectively, scale transformation that may assume the same increase tendency is required, so that the x-axis is transformed into a ratio of Po which is an initial pressure value to P, and the y-axis is transformed into a ratio of Po which is an initial ADC value to an ADC value. The scale for each axis is transformed into a log scale and compared, and is expressed as − so that a rise may be intuitively recognized as an increasing tendency. Finally, as the result of transforming the x-axis into −log(Pressure/Po) value and transforming the y-axis into −log(Intensity/Io) value, it is found that they are changed into the same pattern for two types of light. Since it is confirmed that pressure is changed in the same pattern, it is possible to deduce a correction function that is applicable to both types of light having two wavelengths by linear interpolation in the state of transforming the scale.

FIG. 9 shows the result of obtaining a slope value that fits for the y-axis value with respect to the x-axis by performing linear interpolation in the graph of FIG. 8, which is a scale-changed graph. The scale-changed graph shows linear distribution, and the linear slope gets the value of 0.76 when the linear interpolation is performed regardless of the wavelength of light. The value of 0.76 that is the slope obtained by the interpolation may be determined as the correction function through inverse transformation from a SCALE during the interpolation to a SCALE of the ADC value acquired from the detection module.

In the above-described embodiment, the method of simultaneously performing the interpolation using detection data acquired when both types of light having two wavelengths are applied has been described. However, correction may be performed by individually deducing the correction function for each wavelength.

Meanwhile, the signal processing unit 40 may be provided with an algorithm by loading and applying a correction value from a correction value table generated according to the pressure and wavelength applied on the basis of the correction function. In this case, the signal processing unit 40 may be configured to directly perform correction using a specific value matched without the calculating process of a separate function.

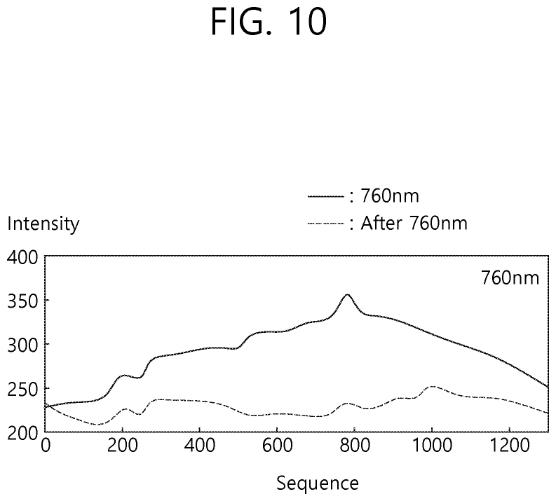

Hereinafter, the result of applying the correction function to the detection optical data will be described with reference to FIGS. 10 to 13.

FIG. 10 is a graph illustrating a result of performing correction when a light source having the wavelength of 760 nm is used. As shown in the drawing, although not shown in the drawing, the applied pressure is experimental data when it is applied differently according to the sequence. Here, it is shown that the acquired ADC value has a large deviation. In contrast, in the case of performing correction using the correction function, it can be seen that the value of ADC is maintained within a predetermined range even if pressure is continuously changed.

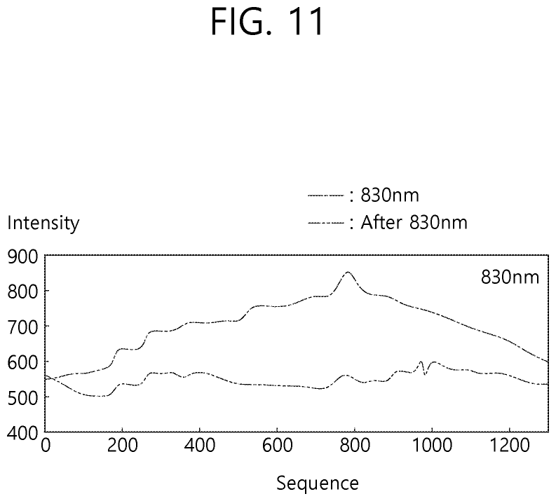

FIG. 11 is a graph illustrating a result of performing correction when a light source having the wavelength of 830 nm is used. In the case of applying the correction function in the same manner as the process of FIG. 10, it can be seen that the range of fluctuation is reduced compared to data before the correction, and the obtained ADC value is maintained in a predetermined range.

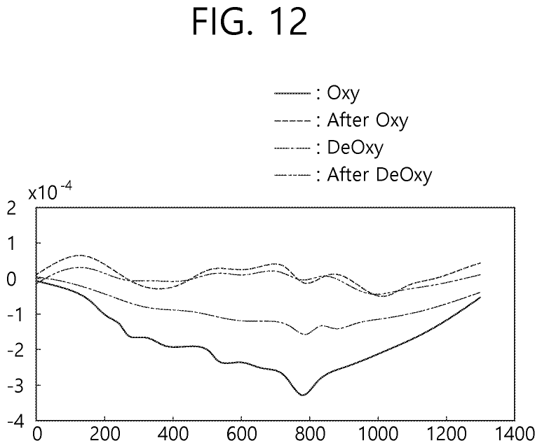

FIG. 12 is a graph before and after the correction of Oxy and Deoxy values. This drawing illustrates the result of calculating the Oxy hemoglobin concentration (Oxy) and the Deoxy hemoglobin concentration (Deoxy) using the Modified Beer-Lambert Law on the basis of the detection optical data shown in FIG. 6. The Oxy and Deoxy values show changes in values relative to initial values, so that the y-axis is shown in an arbitrary unit. In the graph after the correction function is applied, it can be confirmed that variance caused by a change in pressure is greatly reduced as compared with the initial value.

Since the data obtained according to the change of pressure includes noise, the data may be interpreted as having a change in blood flow. However, the value obtained by correcting the data and removing the noise is maintained at a certain level. This may be accepted as a reliable measurement result for a condition where blood flow is not changed in the phantom.

Meanwhile, although not shown in the drawing, the signal processing unit 40 may be configured to include an algorithm for determining acquired data as an error value when the pressure is measured to be equal to or less than a predetermined level. Here, when the pressure is measured to be equal to or less than the predetermined level, the probe 10 is detached from the tissue, which means that the measurement of the data is meaningless. In this case, the data is acquired from the detection module 12 but is determined as the error value, so that it is possible to enhance accuracy. Meanwhile, in the case of having a plurality of probes 10, pressure is determined for each module, so that it is possible to determine whether the data is the error value or not.

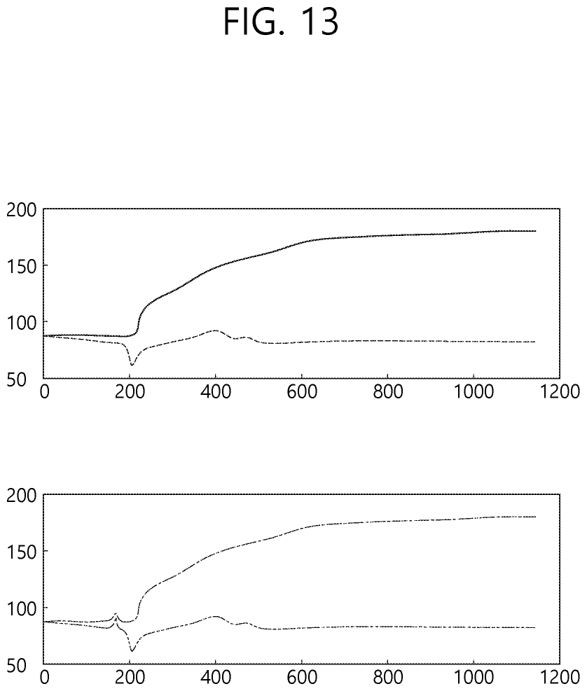

FIG. 13 is a graph illustrating detection values before and after correction when interpolation is performed according to a quadratic interpolating polynominal. FIG. 13(a) and FIG. 13(b) show the results of performing different experiments, respectively. The upper graph of each drawing shows the detection optical data on light having the wavelength of 760 nm, and the lower graph of each drawing shows the detection optical data on light having the wavelength of 830 nm. The accuracy when acquiring the correction function by the quadratic interpolation may be slightly higher than the accuracy by the linear interpolation. It can be seen that noise due to pressure is greatly reduced even when pressure conditions vary and wavelengths are different.

In the above-described embodiment, the method of simultaneously performing the interpolation using detection data acquired when both types of light having two wavelengths are applied has been described, but correction may be performed by individually deducing the correction function for each wavelength.

Furthermore, in the above-described embodiment, the phantom is used to acquire the correction function, but a test for correction is directly performed on a patient, data due to a change in pressure is acquired, and the correction function is acquired according to a patient. However, in this case, this is preferably performed in environment or under conditions where the blood flow rate of a patient is maintained.

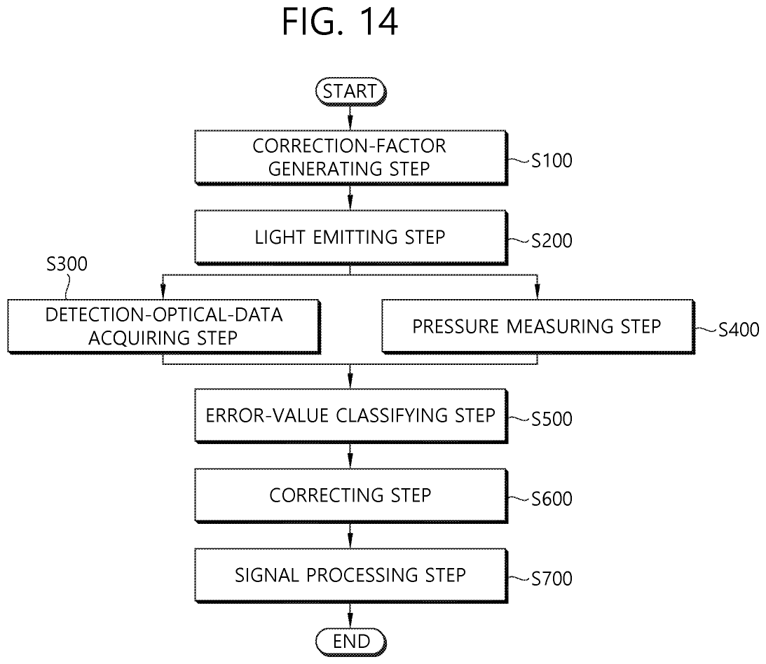

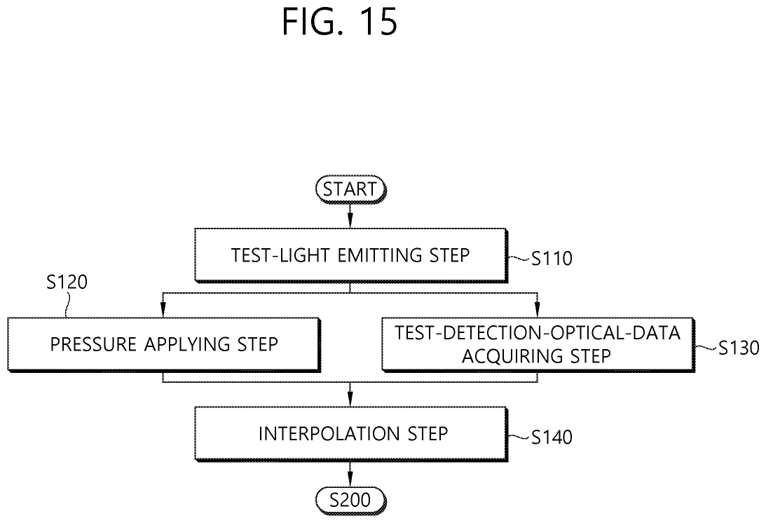

FIG. 14 is a flowchart illustrating a blood flow measuring method in accordance with another embodiment of the present disclosure, and FIG. 15 is a detailed flowchart illustrating a step of generating a correction function.

As shown in the drawing, a blood flow measuring method having a function of correcting noise due to pressure according to the present disclosure may include a correction-function generating step S100, a light emitting step S200, a detection-optical-data acquiring step S300, a pressure measuring step S400, an error-value classifying step S500, a correcting step S600, and a signal processing step S700.

Prior to performing the present disclosure, a step of wearing or contacting the blood flow measuring apparatus on or with a target tissue that is to be measured should be preceded. In order to measure a blood flow rate in a noninvasive manner, the light source module 11, the detection module 12, and the pressure measurement module 13 may come into contact with the skin. For example, in order to measure the blood flow of the brain, the blood flow measuring apparatus may be worn on the head.

The correction-function generating step S100 is the step of generating the correction function for correcting a value recognized as noise when a change in pressure occurs during the measurement of the blood flow rate. The correction-function generating step S100 may be selectively performed after a person to be tested wears the blood flow measuring apparatus. The correction-function generating step S100 may include a test-light emitting step S110, a pressure applying step S120, a test-detection-optical-data acquiring step S130, and an interpolation step S140.

The test-light emitting step S110 is the step of emitting near-infrared light used for measuring blood flow to tissue so as to calculate the correction function. The test light may use light of a wavelength that is used when the blood flow is measured, and may use the same light source.

The pressure applying step S120 is the process of artificially applying pressure to tissue that is to be tested. A user applies appropriate pressure to any one of probes or a plurality of probes 10. For instance, in a state where a user wears the blood flow measuring apparatus, the user may press with the finger to generate appropriate external force and thereby generate pressure.

The test-detection-optical-data acquiring step S130 is the step of acquiring data in a state where pressure is applied. The test-detection-optical-data acquiring step S130 may measure a value such as an ADC value or an intensity in the same manner as when measuring the blood flow data.

The interpolation step S140 corresponds to the step of calculating the correction function required for correction using the acquired data. In the interpolation step S140, the correction function is acquired through the scale transformation and interpolation of the measured detection optical data using the acquired ADC value, assuming that there is no change in blood flow.

As such, if a patient individually acquires the correction function, it is possible to overcome errors caused by a difference among individuals and obtain a precise result value.

After the correction function is acquired, steps S200 to S600 of measuring an actual blood flow rate are performed.

When the blood flow rate is measured, the steps of emitting light, measuring pressure, and detecting light may be performed for a predetermined period.

The light emitting step S200 is the step of emitting the near-infrared light to the tissue through the light source module. In the light emitting step S200, one or more types of light may be selected from types of light having different wavelengths and then may be emitted. In the case of emitting different types of light, light may have different wavelengths to represent different properties among the physical properties of the tissue.

The detection-optical-data acquiring step S300 corresponds to the step of receiving light that returns to the outside of the tissue after light emitted to the tissue is scattered, reflected or the like. The detection optical data may be performed using an electrical element such as the photodiode.

The pressure measuring step S400 is the step of continuously measuring pressure and generating pressure measuring data while light is emitted and detected. Pressure acting between the probe 10 and a surface of the tissue is measured.

The error-value classifying step S500 is configured to exclude corresponding data from blood-flow determination when it corresponds to the error value in pressure measuring step S400. To be more specific, when pressure is beyond a predetermined range, excessively strong external force acts from the outside, thus causing shocks. On the contrary, when pressure is within a predetermined range, a substrate of the blood flow measuring apparatus is spaced apart from a skin surface. In this case, since the measurement result itself is not accurately reflected, it is preferable to exclude this result. Therefore, when the acquired data is determined as the error value and is finally displayed, data excluding this value is displayed.

The correcting step S600 corresponds to the step of applying the acquired correction function to the detection optical data and removing the effect of noise due to pressure. Pressure measurement data is used for each part of the tissue that is to be tested, and the correction function due to the pressure is reflected in the detection optical data, thus correcting the result due to the noise.

The signal processing step S700 corresponds to the step of transforming the corrected detection optical data into a significant result. The detection optical data may process a signal to be transformed into the Oxy hemoglobin concentration (Oxy) and Deoxy hemoglobin concentration (Deoxy) values using the algorithm for transforming the data into a value that may grasp the metabolism, for example, the Modified Beer-Lambert Law.

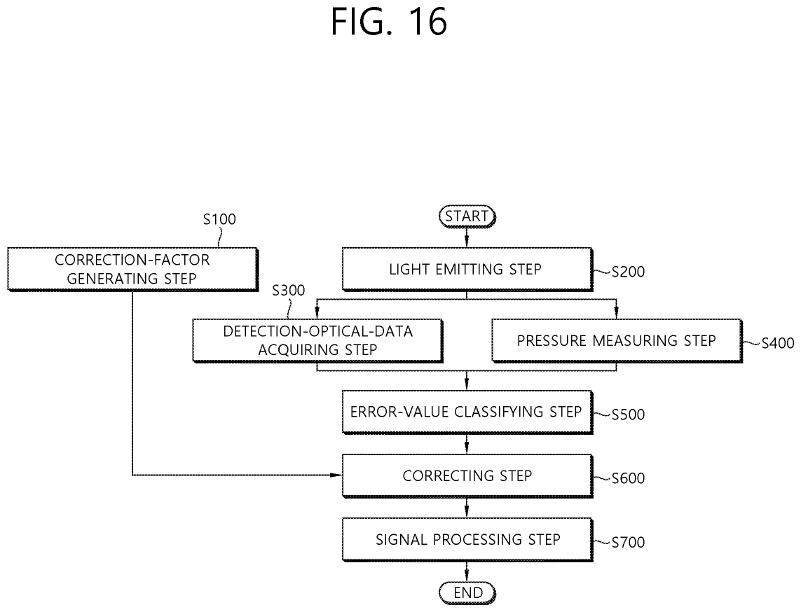

FIG. 16 is a flowchart illustrating a blood flow measuring method in accordance with another embodiment of the present disclosure.

This embodiment may include the same components as the above-described embodiment. In order to avoid a duplicated description, the description of the same components will be omitted and only different components will be described.

As shown in the drawing, in this embodiment, the correction function may be previously deduced and used before measuring the blood flow rate. The preset correction function may be deduced using the phantom.

The deduction using the phantom may be performed in the same manner as the correction-function deducing step of the above-described embodiment except that the phantom is used as a target. In this embodiment, since it may be selected within the range of a preset correction function and may be applied immediately, separate test time is not required, thus minimizing the waste of resources.

As described above, a blood flow measuring apparatus and method having a function of correcting noise due to pressure according to the present disclosure can calculate a correction function in consideration of the effect of pressure and can correct measured blood flow by reflecting the correction function. Therefore, the present disclosure has an advantage that the accuracy and reliability of a noninvasive blood flow test can be improved.

1. A blood flow measuring apparatus having a function of correcting noise due to pressure, comprising:
a light source module configured to come into contact with tissue to emit light;
a detection module configured to detect light that has undergone at least one process of scattering, absorption, and reflection while moving along the tissue;
a pressure measurement module configured to measure pressure acting on the tissue; and
a signal processing unit configured to correct noise generated as the pressure acts during the detection of the light, and thereby process a signal received from the detection module.

2. The blood flow measuring apparatus of claim 1, wherein the signal processing unit corrects the signal received from the detection module using the correction function due to the pressure.

3. The blood flow measuring apparatus of claim 1, wherein the signal processing unit performs correction using a correction value matching with the pressure from a correction value table.

4. The blood flow measuring apparatus of claim 2, wherein the correction function is acquired by interpolating detection optical data acquired when the pressure is changed in a state where light is emitted to the tissue with the light source module.

5. The blood flow measuring apparatus of claim 4, wherein the correction function is acquired by interpolating detection optical data acquired by changing pressure when two types of light having different wavelengths are emitted to the tissue.

6. The blood flow measuring apparatus of claim 5, wherein the light is selected in a near-infrared area, and the signal processing unit transforms data on a light intensity into Oxy hemoglobin concentration and Deoxy hemoglobin concentration to grasp metabolism of the tissue on the basis of the detection optical data.

7. The blood flow measuring apparatus of claim 5, wherein the signal processing unit performs interpolation through transformation of at least one of variance for an initial measurement value or log scale so that change tendencies depending on a change in pressure are synchronized for values detecting light having different wavelengths.

8. The blood flow measuring apparatus of claim 2, wherein the correction function deduces −log(Pressure/Po) value from the pressure data, deduces −log(Intensity/Io) value from the detection optical data, and is determined by interpolation according to distribution of the −log(Pressure/Po) value and the −log(Intensity/Io) value.

9. The blood flow measuring apparatus of claim 8, wherein the correction function acquires the −log(Pressure/Po) value and the −log(Intensity/Io) value by linear interpolation or quadratic interpolation.

10. The blood flow measuring apparatus of claim 2, wherein the correction function is calculated on the basis of detection data acquired according to pressure in a state where the light is emitted to a phantom.

11. The blood flow measuring apparatus of claim 2, wherein the light source module, the detection module, and the pressure measurement module constitute one probe, and a plurality of probes is connected to each other to surround a head.

12. The blood flow measuring apparatus of claim 1, wherein the light source module, the detection module, and the pressure measurement module are provided on a surface of the probe to come into contact with the tissue.

13. The blood flow measuring apparatus of claim 2, wherein the signal processing unit determines a value acquired from the detection module as an error value, when a value measured in the pressure measurement module is equal to or less than a reference value.

14. A blood flow measuring method having a function of correcting noise due to pressure, comprising:
a step of emitting light while coming into contact with tissue;
a step of acquiring detection optical data by detecting light that has undergone at least one process of scattering, absorption, and reflection while moving along the tissue;
a step of measuring pressure acting on the tissue; and
a correcting step of reflecting a correction function depending on the pressure to the detection optical data so as to remove noise of the detection optical data due to the pressure.

15. The blood flow measuring method of claim 14, wherein the correction function is calculated by performing a correction-function generating step, and the correction-function generating step is acquired by performing:
a test-light emitting step of emitting light to the tissue;
a step of changing pressure applied to the tissue;
a step of acquiring test detection optical data according to a change in the pressure; and
an interpolation step of performing interpolation from the detection optical data and the pressure data.

16. The blood flow measuring method of claim 15, wherein the interpolation step deduces −log(Pressure/Po) value from the pressure data, deduces −log(Intensity/Io) value from the detection optical data, and performs interpolation according to distribution of the −log(Pressure/Po) value and the −log(Intensity/Io) value.

17. The blood flow measuring method of claim 16, wherein the interpolation step determines the −log(Pressure/Po) value and the −log(Intensity/Io) value with the correction function acquired by linear interpolation or quadratic interpolation.

18. The blood flow measuring method of claim 14, wherein the step of emitting light uses light in a near-infrared area, and the method further comprising:
after the correcting step, a signal processing step of transforming data on a light intensity into Oxy hemoglobin concentration and Deoxy hemoglobin concentration to grasp metabolism of the tissue on the basis of the detection optical data.

19. The blood flow measuring method of claim 14, wherein the correction function is calculated by performing a correction-function generating step, and is calculated by performing:
a test-light emitting step of emitting light to a phantom;
a step of changing pressure applied to the phantom;
a step of acquiring test detection optical data according to a change in the pressure; and
an interpolation step of performing interpolation from the detection optical data and the pressure data.

20. The blood flow measuring method of claim 14, further comprising:
an error-value classifying step of determining the detection optical data as an error value, when a measured pressure value is less than a reference value.
